(19) United States
(12) Patent Application Publication
(10) Pub. No.: US 2021/0045677 A1
(43) Pub. Date: Feb. 18, 2021

(54) Apparatus and Methods for Determining and Responding to the Location, Orientation, and Respiration of a Person

(71) Applicant: Hill-Rom Services, Inc., Batesville, IN (US)

(72) Inventors: David L. Ribble, Indianapolis, IN (US); Kirsten M. Emmons, Batesville, IN (US); Yongji Fu, Harrison, OH (US); Craig M. Meyerson, Syracuse, NY (US)

(21) Appl. No.: 16/537,963

(22) Filed: Aug. 12, 2019

(51) Int. Cl. A61B 5/00; A61B 5/08

(52) U.S. Cl. CPC A61B 5/4818; A61B 2562/0204; A61B 5/4809; A61B 5/08

(57) ABSTRACT

A system for addressing sleep disordered breathing in a person, comprises an acoustic sensor, a processor, and a support apparatus for supporting the person. The support apparatus includes or is receptive to receiving a turn effector. The system also includes processor executable instructions which, when executed by the processor, cause the system to 1) determine upper airway orientation of the person based on information from the acoustic sensor, 2) determine, based on the information from the acoustic sensor, if the person is experiencing sleep disordered breathing, and 3) if the person is experiencing sleep disordered breathing, command operation of the turn effector to act on a component of the support apparatus thereby changing the orientation of the upper airway of the person from an initial orientation to a target orientation. The target orientation is an orientation more conducive to relieving the sleep disordered breathing than is the initial orientation.

### TECHNICAL FIELD

This application relates to an apparatus or system and associated method for using acoustic information to determine parameters such as the location, upper airway orientation, and respiration state of a person and to respond to the detected parameters when appropriate. One application of the apparatus and method is in a hospital setting where the person is a patient and knowledge of the parameters may be used to enhance the patient's well-being.

### BACKGROUND

In a hospital setting it can be important to determine whether or not a patient is in his bed. A patient who has exited or egressed from the bed, and who is not authorized to do so without a caregiver being present, may have placed himself at risk of injury. When a patient is in the bed, knowledge of his position can be helpful for predicting if he will attempt to exit the bed or is in the act of exiting. Even a patient who is authorized to exit the bed should be monitored for any absence that seems unreasonably lengthy. In addition, knowledge of the orientation of a person's upper airway can help identify if the patient suffering an apneic episode or is at risk of apnea.

Traditionally, determinations of patient's position and orientation on a bed and the parameters of his upper airway have relied on optical sensors or measurements of patient weight or motion using, for example, load cell sensors and pressure sensors. Despite the merits of such arrangements further developments are desirable, particularly those that can detect not only patient presence/absence and egress status, but also whether or not the patient is suffering from sleep disordered breathing such as apnea.

### SUMMARY

A system for addressing sleep disordered breathing in a person, comprises an acoustic sensor, a processor, and a support apparatus for supporting the person. The support apparatus includes or is receptive to receiving a turn effector. The system also includes processor executable instructions which, when executed by the processor, cause the system to 1) determine upper airway orientation of the person based on information from the acoustic sensor, 2) determine, based on the information from the acoustic sensor, if the person is experiencing sleep disordered breathing; and 3) if the person is experiencing sleep disordered breathing, command operation of the turn effector to act on a component of the support apparatus thereby changing the orientation of the upper airway of the person from an initial orientation to a target orientation. The target orientation is an orientation more conducive to relieving the sleep disordered breathing than is the initial orientation.

### BRIEF DESCRIPTION OF THE DRAWINGS

The foregoing and other features of the various embodiments of the systems and methods described herein will become more apparent from the following detailed description and the accompanying drawings in which:

FIG. 1 is a schematic plan view of a hospital room having a single patient bed, other points of interest including a chair, toilet and sink, and acoustic sensors such as microphones distributed throughout the room.

FIG. 2 is view similar to FIG. 1 with a different distribution of microphones.

FIG. 3A is a schematic head end elevation view of a hospital bed having a mattress and turn effectors in the form of turn bladders and showing one of the bladders in a deflated state (solid lines) and in an inflated state (dashed lines) and showing a portion of the mattress with solid and dashed lines depending on the state of the turn bladder.

FIG. 3B is a schematic plan view taken in direction 3B-3B of FIG. 3A showing a bed architecture which includes a deck and four turn bladders.

FIG. 4 is a view similar to FIG. 3A in which the deck includes left and right segments hinged to each other and actuators in lieu of the bladders of FIGS. 3A and 3B.

FIG. 5 is a block diagram showing one mode of operation of an acoustically based system for determining patient presence or absence in response to inputs from the microphones of FIG. 1 and, if present, his perceived location in the room.

FIG. 6 is a schematic view showing locating of a sound source by triangulation.

FIG. 7 is a block diagram similar to FIG. 5 but also including a filtering step which may be either a filtering out of non-patient sounds or an admission of only patient related sounds.

FIG. 8 is a block diagram illustrating the use of acoustic information from the microphones of FIG. 2 to pre-emptively address the possibility of apnea.

FIG. 9 is a set of schematic head end elevation views of a patient lying on a support surface in order to illustrate the concepts of shoulder supine and airway supine.

FIG. 10 is a view along a person's sagittal plane showing the person's upper airway.

FIG. 11 is a diagram showing one example of responses provided by a turn effector in response to a patient's state of airway supineness.

FIG. 12 is a block diagram similar to FIG. 8 including a feedback loop.

FIG. 13 a diagram illustrating the use of acoustic information to address an actual occurrence of sleep disordered breathing by turning the patient.

FIG. 14 is an illustrative graph showing the sound of snoring as oscillations separated by periods of normal breathing.

FIG. 15 is an illustrative graph showing a breathing state typical of apnea.

FIG. 16 is a diagram showing a variant of FIG. 13 in which turning of the patient is conditional.

FIG. 17 is a block diagram showing a variant of FIG. 16 in which an explicit distinction is made between snoring and apnea.

FIG. 18 is a plan view similar to FIG. 2 showing a sound generator used to improve system performance.

FIG. 19 is a plan view similar to FIG. 2 in which the room includes two patient beds.

FIGS. 20A, 20B and 20C are block diagrams showing method steps for addressing sleep disordered breathing.

FIG. 21A is a schematic plan view of a first embodiment of a two person bed, such as a residential bed, whose occupants may benefit from the apparatus and method described herein.

FIG. 21B is a view in direction 21B-21B of FIG. 21A.

FIG. 21A is a schematic plan view of a second embodiment of a two-person bed.

FIG. 22B is a view in direction 22B-22B of FIG. 22A.

FIG. 22C is a view in direction 22C-22C of FIG. 22A.

FIG. 23A is a schematic plan view of a third embodiment of a two-person bed.

FIG. 23B is a view in direction 23B-23B of FIG. 23A.

### DETAILED DESCRIPTION

The present invention may comprise one or more of the features recited in the appended claims and/or one or more of the following features or combinations thereof.

In this specification and drawings, features similar to or the same as features already described may be identified by reference characters or numerals which are the same as or similar to those previously used. Similar elements may be identified by a common reference character or numeral, with suffixes being used to refer to specific occurrences of the element. Examples given in this application are prophetic examples.

The following are incorporated herein by reference:
1. US Patent Application Publication 2015/033,5507
2. U.S. Design Pat. No. D779,236
3. US Patent Application Publication 2014/034,5060
4. International Application Publication WO 2013/177,338.

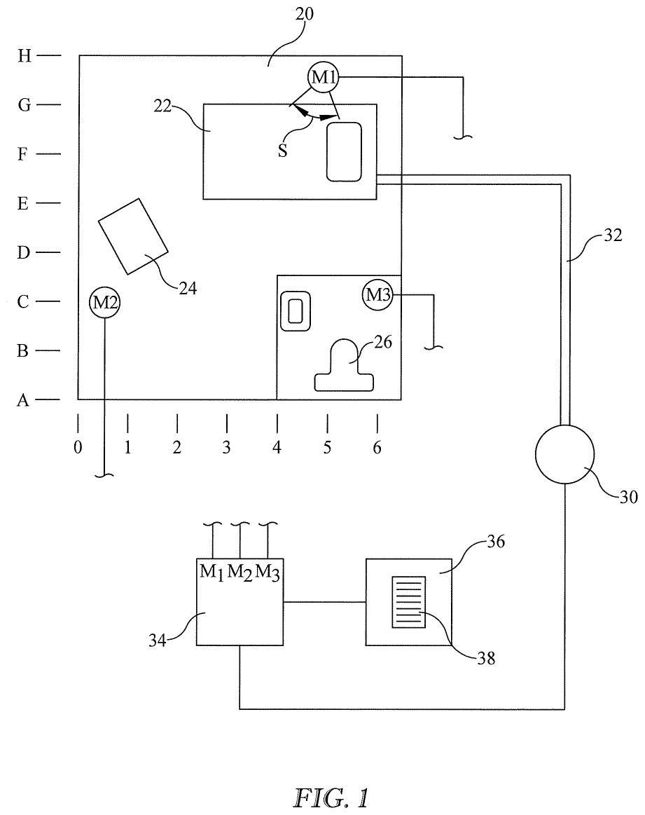

FIG. 1 is a schematic plan view of a hospital room 20. Points of interest in the room include a bed 22 (or other support apparatus) for a patient, a chair 24 and a toilet 26. The room includes three microphones or other sound detectors, M1, M2, M3. Microphone M1 is near the bed; microphone M2 is near the chair; microphone M3 is near the toilet.

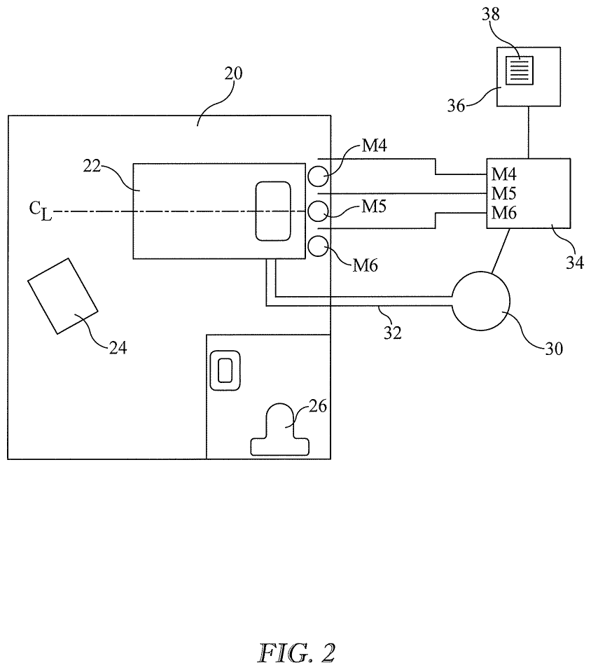

FIG. 2 is a schematic plan view of a hospital room 20 similar to that of FIG. 1. The room of FIG. 2 includes three microphones or other sound detectors, M4, M5, M6 laterally spaced from each other along the head end of the bed with microphone M5 laterally aligned with the longitudinally extending centerline CL of the bed.

In connection with the subject matter described herein microphone arrangements other than those shown in FIG. 1 and FIG. 2 may be used. In some embodiments the microphones may be directional microphones, i.e. microphones that are most sensitive to sound originating from a particular angular sector such as sector S of FIG. 1.

FIGS. 1-2 also show an air pump 30, an air supply line 32 extending therefrom, a processor 34, and a memory 36 which holds processor executable instructions 38. These components are discussed in more detail below. Due to the scale of FIGS. 1-2 and other similar drawings, the pump, processor, and memory are shown outside room 20. In practice the pump, processor and memory may be elsewhere, including in the room. The processor executable instructions, when executed by the processor, cause the system or apparatus to carry out certain actions. Equivalently, the processor, acting in accordance with the instructions, causes the system or apparatus to carry out those actions.

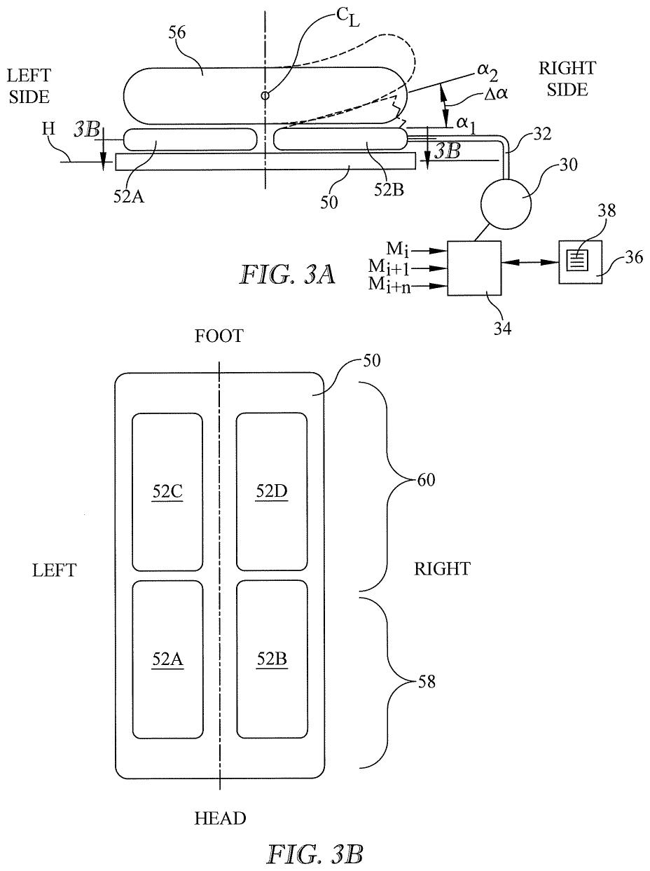

FIGS. 3A-3B are a schematic head end elevation view and a plan view of a representative bed 22. The bed extends longitudinally from a head end to a foot end and laterally from a left side to a right side. Left and right are taken from the perspective of a supine occupant of the bed. The bed includes a frame having a deck 50, inflatable and deflatable turn bladders 52, and a mattress 56 resting on the deck. The bed, including the frame and mattress, may be thought of as having sections corresponding to anatomical features of an occupant's body such as a torso section 58 corresponding approximately to the occupant's torso and head when the occupant is positioned properly on the mattress, and a lower body section 60 corresponding approximately to the occupant's buttocks, legs and feet when the occupant is positioned properly on the mattress.

Turn bladders 52 are operable (inflatable and deflatable) in order to rotate the mattress or a portion thereof to the left or right thereby turning or rotating a patient occupying the bed, or a portion of his body, to his left or right. Therefore, the bladders are referred to as turn effectors. Each bladder may be operable independently of the other bladders. The architecture of FIGS. 3A-3B includes four turn bladders, torso section bladders 52A, 52B corresponding approximately to a patient's torso, and lower body section bladders 52C, 52D corresponding approximately to the patient's legs.

FIG. 3A also shows air pump 30, fluid supply line 32, processor 34, memory 36, and instructions 38 of FIGS. 1-2. The microphone inputs in FIG. 3A are labeled as Mᵢ, Mᵢ₊₁, Mᵢ₊ₙ. FIG. 3A shows that fluid supply line 32 extends from the pump to the bladders 52, however the line to bladder 52B is the only one illustrated. Bladders 52, pump 30, and supply line 32 comprise a turn effector assembly.

In operation the processor receives input signals such as signals from the microphones representing the sounds detected by each microphone. The processor issues commands based on the input signals and the processor executable instructions. In connection with the system and apparatus described herein the processor issues commands to air pump 30 causing the pump to operate to inflate and deflate selected turn bladders. For example if the instructions call for inflation of a particular bladder or bladders in response to the input signals, the controller operates the pump to supply pressurized air to the bladder(s). Each illustrated bladder 52 has a bellows-like construction so that it expands more at its laterally outboard extremity than at its laterally inboard extremity. The illustrated inflation of bladder 52B would turn a supine patient's torso to the patient's left, but is not intended to turn the patient's legs. Nevertheless his legs may also turn somewhat to the left to the extent that his legs naturally turn along with his torso. Intentional leftward turning of the patient's legs would be carried out by inflating bladder 52D. The angle through which a portion of the patient's body is rotated will mimic bladder rotation angle Δα but will not likely be identical to Δα.

If the instructions call for deflation of a particular bladder or bladders in response to the input signals, processor 34 may operate the pump to suction air from the bladder. In one alternative arrangement the bladders may have a vent valve, and the processor may issue a command to open the vent valve thereby venting the bladder to the surroundings. Alternatively, the instructions may cause the processor to issue a communication to a caregiver advising the caregiver of the need to inflate or deflate the bladders so that the caregiver can operate the pump manually.

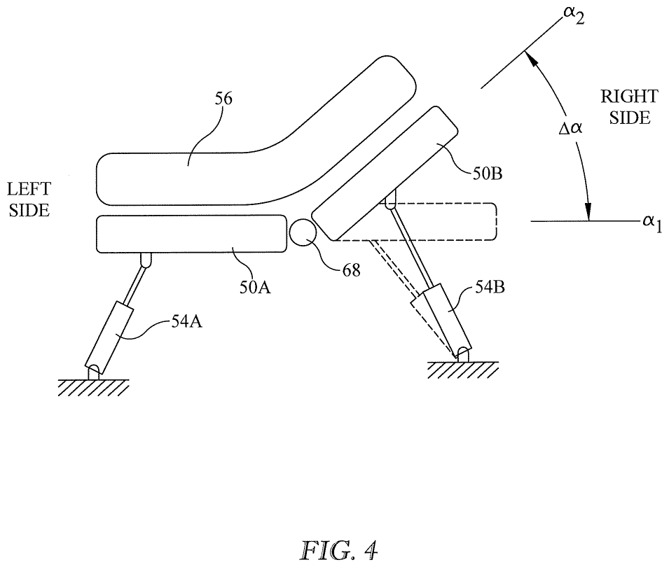

Other bed architectures may be employed to accomplish patient rotation. For example as seen in FIG. 4 deck 50 may include left and right segments 50A, 50B connected to each other at a hinge 68. (The term “segment” may be similarly applied to the mattress and to the bed as a whole.) The turn effectors are actuators 54. The processor issues commands to operate an actuator. In response the commanded actuator rotate one segment or the other of the deck about the hinge to turn the patient. In another embodiment, not illustrated, the deck is not hinged (as in FIG. 3A) and the turn effector tilts the entire deck about a longitudinally extending axis. To the extent that turning of the patient is required, any arrangement capable of providing the required range of turning may be employed. In general, the turn effector acts to turn or tilt some component or components of the deck (i.e. to change the orientation of the component or components) in order to turn the patient to the left or right.

The turn effectors described above are turning subsystems that are integrated into the bed by reason of being features incorporated into or included with the bed frame and/or mattress at the time of manufacture. Nonintegrated turning subsystems may also be used to provide the turn capability. A nonintegrated turning subsystem is one that is not incorporated into the frame or mattress at the time of manufacture. The nonintegrated turning subsystem is available to the end user and can be installed on the bed on an as-needed basis to achieve the turning capability. A bed (or the frame or mattress thereof) which is compatible with a nonintegrated turning subsystems is referred to as being receptive to receiving the turning subsystem. Some beds may be expressly designed to be receptive. Other beds may be inherently receptive despite not having been intentionally designed to accommodate a turning subsystem. Yet other beds may be modifiable to a receptive configuration despite not having been designed or manufactured to be receptive to receiving a turning subsystem.

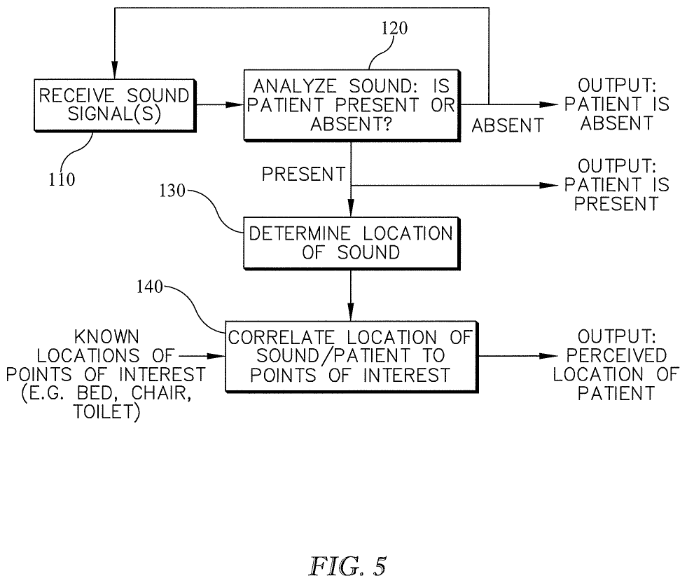

The block diagram of FIG. 5 shows one mode of system operation in response to inputs from microphones M1, M2, M3 of FIG. 1. The mode of operation of FIG. 5 is to determine at least if the patient is present or absent. The diagrammed mode of operation may also determine the location of the patient and may determine his location relative to various points of interest. At block 110 the system receives signals from the microphone representing the sounds detected by the microphones. Because the signals output from the microphone represent the sounds detected by the microphone they may be referred to herein as sound(s) rather than signal(s). In this and other embodiments any sound or sounds suitable for the intended purpose may be employed, e.g. sounds of patient breathing, snoring, moving about on the bed, or moving about in the room.

At block 120 the processor analyzes the sound profile from the microphones to determine if the patient is present or absent. In one example the sound profile is the time signature of the sound (amplitude vs. time) and the processor carries out the analysis by comparing the detected time signature to candidate signatures stored in memory 36 that correspond to human attributable sounds such as breathing, snoring or speaking. In another example the sound profile is the sound spectrum of the detected sound, i.e. its frequency harmonics and their amplitudes. The processor carries out the analysis by comparing the detected spectrum to candidate spectrums stored in memory 36 that correspond to human attributable sounds such as breathing, snoring or speaking. If the detected sound profile matches one of the stored profiles within a prescribed tolerance, the patient is presumed to be present. Otherwise the patient is presumed to be absent and the processor branches back to block 110. Either way the processor may produce a “Patient is Absent” signal or “Patient is Present” signal to be delivered to a suitable destination such as a local or remote display monitor.

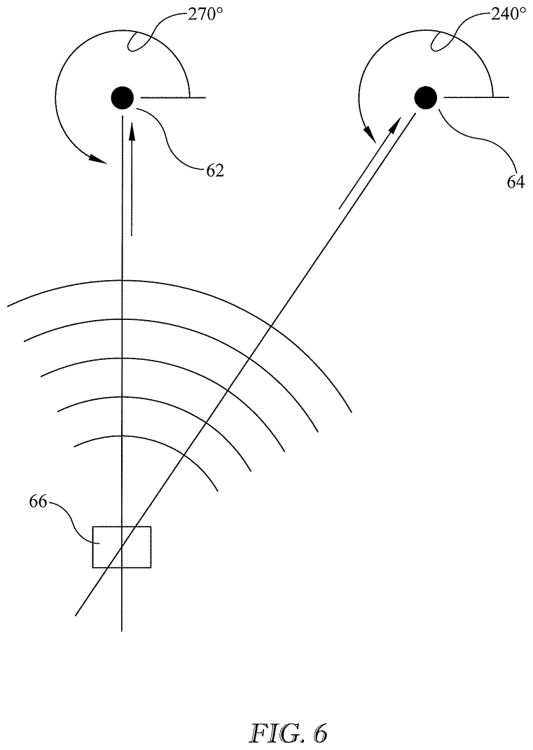

If a patient is present the operation of the system may include the additional step at block 130 of determining the location of the patient originated sound. Location may be determined in any suitable way, for example by assessing the bearing from each microphone to the patient originated sound and triangulating to ascertain the actual location of the patient as seen in FIG. 6. FIG. 6 shows a first microphone 62 perceiving the sound source at a bearing of 270 degrees, and a second microphone 64 perceiving the sound source 66 at a bearing of 240 degrees. The processor determines by triangulation that the sound source is at the intersection of the dashed lines.

Returning to FIG. 5, at block 140, the processor correlates the estimated location of the sounds, and therefore the estimated or perceived location of the patient, to the known locations of one or more points of interest, for example physical objects such as bed 22, chair 24 and toilet 26. Another example of points of interest are Cartesian coordinates of the room defined by the alphabetic scale (A through H) and numeric scale (0 through 6) seen in FIG. 1. The system output is a report of the perceived location of the patient. The perceived location is the known location that most closely corresponds to the location determined at block 130.

In one embodiment the correlation at block 140 involves a comparison of relative sound amplitudes detected at the microphones. Table 1 below shows an example of the conclusions that might be reached based on a binary characterization of sound amplitude: HIGH (H) and LOW (L).

TABLE 1

| | Sound | Sound | Sound | Conclusion |
|---|---|---|---|---|
| Row | Volume at | Volume at | Volume at | about Patient |
| Number | Microphone 1 | Microphone 2 | Microphone 3 | Location |
| 1 | L | L | L | Indeter- |
| | | | | minate |
| | | | | or not |
| | | | | present |
| 2 | L | L | H | Toilet |
| 3 | L | H | L | Chair |
| 4 | L | H | H | Indeter- |
| | | | | minate, |
| | | | | or between |
| | | | | chair and |
| | | | | toilet, or |
| | | | | not in the |
| | | | | bed |
| 5 | H | L | L | Bed |
| 6 | H | L | H | Bed or |
| | | | | Toilet, or |
| | | | | between bed |
| | | | | and toilet, |
| | | | | or not in |
| | | | | the chair |
| 7 | H | H | L | Vicinity |
| | | | | of Bed and |
| | | | | Chair |
| 8 | H | H | H | Indeter- |
| | | | | minate |

The interpretation assigned to any given set of relative volumes may be determined by the system designer. For example the relative sound volume of rows 1, 4 and 6 of the table could be interpreted any one of the multiple ways indicated. The ability to assign a definite interpretation to a set of relative sound volumes may be improved by, for example, the use of microphones designed to detect sounds only from a selected direction, by the use of more microphones, by the use of more than two characterizations of sound amplitude, or by some combination of these and/or other measures.

The information obtained may be put to any suitable use. One example is to track the activity of a patient such as time spent in and out of the bed. A lengthy visit to the toilet, or detection at the toilet followed by no detection of the patient anywhere else after a considerable lapse of time may indicate that the patient needs assistance. Detection that the patient is in the bed may trigger the activation of a protocol designed to ensure that the patient does not fall out of the bed or to assess if the patient is making an unauthorized egress from the bed or is attempting to do so. In another example if the patient is absent and is not expected to be, an alert may be generated and delivered to a nurses' station or to a communication device carried by nurses. A similar alert may be generated and delivered if the patient is present but is expected to be elsewhere, for example undergoing an x-ray.

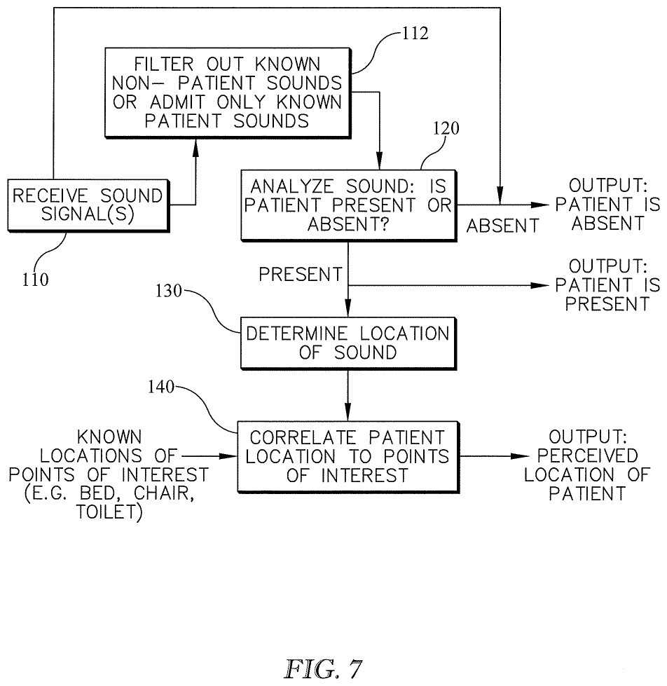

FIG. 7 is a block diagram similar to FIG. 5. The diagram of FIG. 7 includes additional block 112. In one embodiment block 112 filters out sound signals that are not characteristic of a sound a person might make. These are referred to as non-patient sounds. Example non-patient sounds include background noise from a climate control system. In another embodiment block 112 admits to block 120 only those sound signals that are characteristic of a sound a person might make. Block 112 may be used in other block diagrams of the instant application but has not been included in those diagrams in the interest of simplicity.

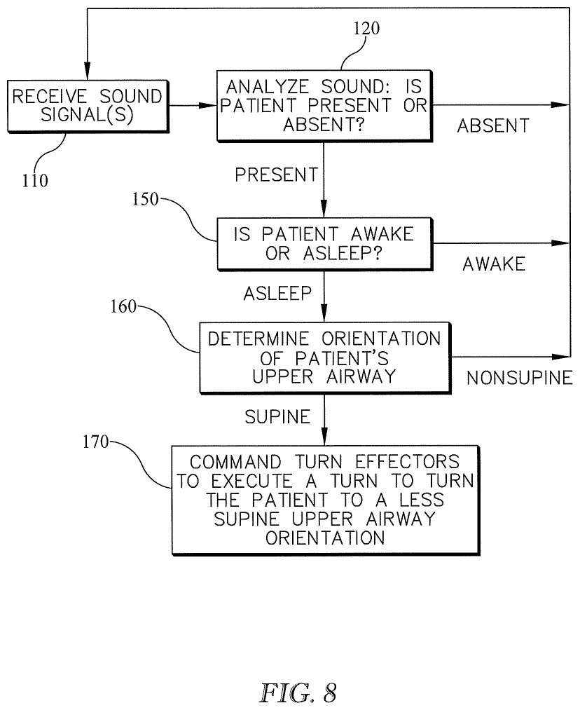

FIG. 8 is a diagram illustrating the use of acoustic information from microphones M4, M5, M6 of FIG. 2 to pre-emptively address the possibility of apnea, which is the cessation of breathing. Apnea diagnosis guidelines may require that the breathing cessation last a minimum period of time (not including normal pauses after inhalation and after exhalation) and/or occur repeatedly, for example more than specified number of occurrences over a specified period of time.

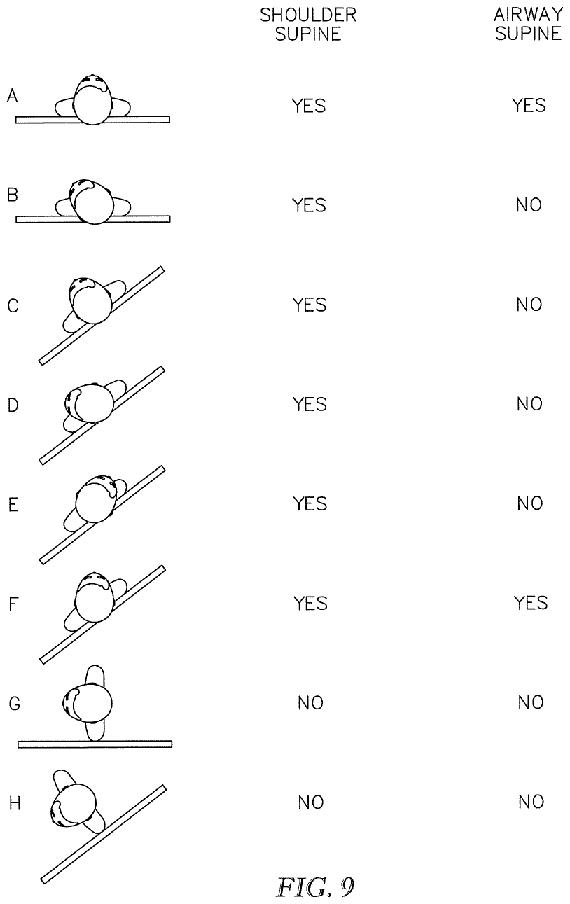

Before proceeding with the discussion of apnea it will be useful to address the notion of “supine”. Referring additionally to FIG. 9, a patient is “shoulder supine” if his shoulders are touching support surface 56 (views A through F). A patient is “airway supine” if his nose and line of sight are directed vertically upwardly, irrespective of the orientation of the support surface (Views A and F only). Unless specified otherwise, the term supine as used in this specification means airway supine. In general, a less supine upper airway orientation is more conducive to preventing or relieving sleep disordered breathing while a more supine upper airway orientation is less conducive to preventing or relieving sleep disordered breathing. Turning a patient to his left or right in order to achieve a less supine upper airway orientation is referred to herein as turn therapy.

Returning to FIG. 8, blocks 110 and 120 are the same as already described. At block 150 the processor determines the patient's state of wakefulness, i.e. if the patient is awake or asleep. The determination of wakefulness can be determined by, for example, detecting the amplitude and/or rate of the patient's respiration, both of which are typically lower in the sleep state than in the awake state unless the patient is in the REM sleep stage. Other options for establishing the patient's state of wakefulness include detecting the patient's voice (a strong indication that the patient is awake) or detecting snoring (an indication that the patient is asleep). These techniques may be used independently of each other or in various combinations to improve the accuracy of the asleep/awake determination.

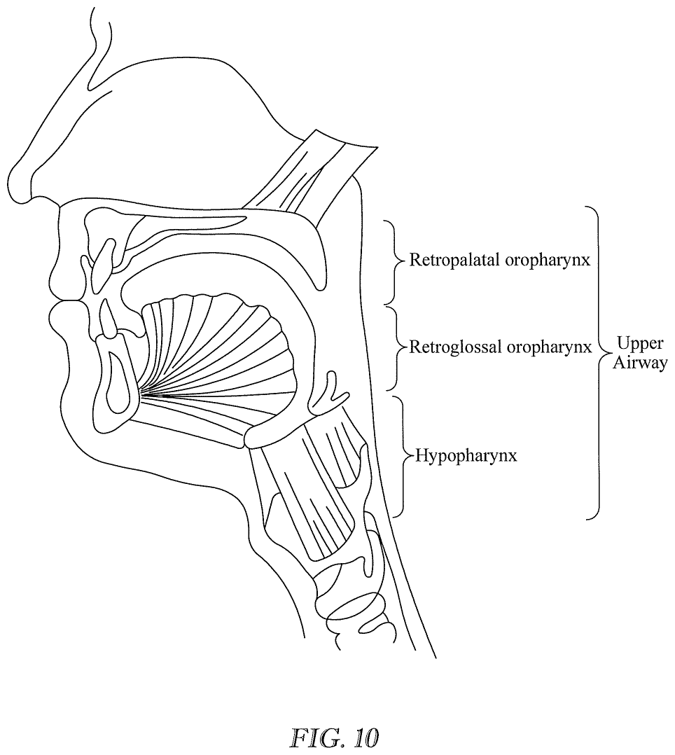

If the patient is awake, an apneic event cannot occur, therefore the processor continues to evaluate the patient's state of wakefulness. If the patient is asleep, the processor advances to block 160 and determines the orientation of the patient's upper airway. Unless specified otherwise, references herein to “airway” mean “upper airway”. Referring to FIG. 10, the upper airway is considered to be the hypopharynx, the retroglossal oropharynx, and the retropalatal oropharynx.

Determination of the orientation of the upper airway can be made by monitoring the sounds detected at the microphones. For example, referring to the microphone distribution of FIG. 2, if the amplitude of the patient's breathing or snoring is symmetric or nearly symmetric with respect to microphone M5 (about equal at M4 and M6 irrespective of the amplitude at M5) the patient's upper airway is supine or approximately supine, which is an orientation not favorable for avoidance of apnea. Therefore the patient is considered to be excessively supine. Accordingly, at block 170 the processor commands the turn effector of the bed to act on a bed component in such a way as to re-orient the patient's upper airway from the initial more supine orientation, to a target, less supine orientation. The change in patient airway orientation is accomplished by commanding the turn effector to execute a left or right turn of Δα degrees. Taking the arrangement of FIG. 3A as an example, the processor commands inflation of a bladder 52 to reorient the top surface of the bladder from a first orientation (e.g. 52A as depicted with a solid line) to a second orientation (e.g. 52A as depicted with a dashed line). The change in orientation is shown as Δα=α2−α1 where α2 and α1 are taken relative to horizontal H. Taking the arrangement of FIG. 4 as an example, the processor commands actuator 54A to extend thereby changing the orientation of deck segment 50B through angle Δα. If the system is sensitive enough to determine that the patient is already turned slightly to the left or slightly to the right, the system will execute a left turn or a right turn respectively. The patient may turn somewhat more or less than the Δα degree turn executed by the bed components.

If the amplitude of the patient's breathing or snoring is moderately asymmetric with respect to microphone M5 (louder at M4 than at M6 or vice versa) the patient's upper airway is nonsupine, but may nevertheless be near enough to supine to cause an unacceptably high risk of an apneic event. Accordingly, at block 170 the processor commands the lateral turn effector of the bed to execute a turn that will orient the patient's upper airway in a less supine orientation, for example by commanding the turn effector to execute a left turn of (Δα)₁ degrees if the existing (pre-turn) orientation of the patient's airway is nonsupine to the left, or a right turn of (Δα)₁ degrees if the existing (pre-turn) orientation of the patient's airway is nonsupine to the right. Because the patient's pre-turn airway orientation is initially somewhat non-supine, the magnitude of (Δα)₁ is less than the magnitude of (Δα)₂ (where (Δα)₂ is the change of orientation applied if the initial orientation of the patient's upper airway is supine or nearly so, as described in the previous paragraph). The patient's upper airway may turn somewhat more or less than the (Δα)₁ degree turn executed by the bed.

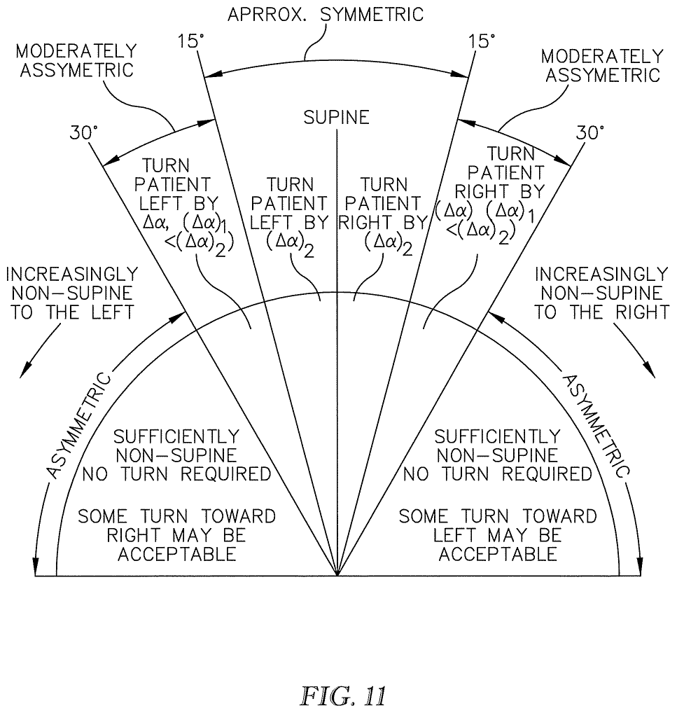

In summary, and referring to FIG. 11, a relatively smaller asymmetry detected by the microphones (including a detection of symmetry) requires a larger correction (Δα)₂ (FIG. 11, sectors labeled “Approximately Symmetric”). A relatively moderate asymmetry detected by the microphones requires a more modest correction (Δα)₁ to the patient's orientation away from supine (FIG. 11, sectors labeled “Moderately Asymmetric”). A high degree of asymmetry may require little or no correction and may even permit a correction toward a supine orientation (FIG. 11, sectors labeled “Highly Asymmetric”). In one embodiment the magnitude of Δα is large enough to achieve a post-turn patient airway orientation of at least about 30 degrees from vertical. Accordingly, the difference between the first and second orientations of the bed components (and therefore between the initial and target orientations of the patient's upper airway) is a function of the first orientation of the bed components, which approximates the initial orientation of the patient's upper airway.

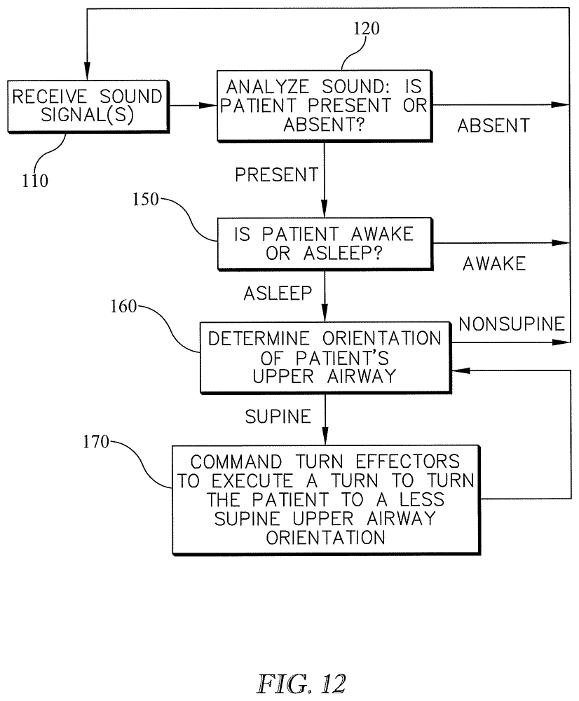

Referring to FIG. 12 the adequacy of the correction can be gauged by assessing how the correction to the patient's airway orientation has affected the asymmetry detected at the microphones. If the post-turn asymmetry detected by the microphones indicates that the patient's upper airway has not turned sufficiently away from supine the processor may command the turn effector to carry out additional corrective turning. This is illustrated in FIG. 12 by the feedback loop extending from block 170 back to block 160. Block 160 continues to use the sound detected at block 110 to update the determination of the patient's airway orientation. Block 170 continues to use the updated determinations to turn the patient in an attempt to put his airway in a beneficial orientation. In summary, the actions at blocks 160 and 170 are continuously ongoing. The embodiment with the feedback loop (FIG. 12), in comparison to the embodiment without the feedback loop (FIG. 8) may require more microphones and/or may be more likely to require instructions 38 that will analyze both amplitude and frequency of the sound detected at block 110.

Referring to FIGS. 8 and 12, it may be possible to dispense with block 120 because a positive determination that a patient is awake or asleep at block 150 ensures that a patient is present.

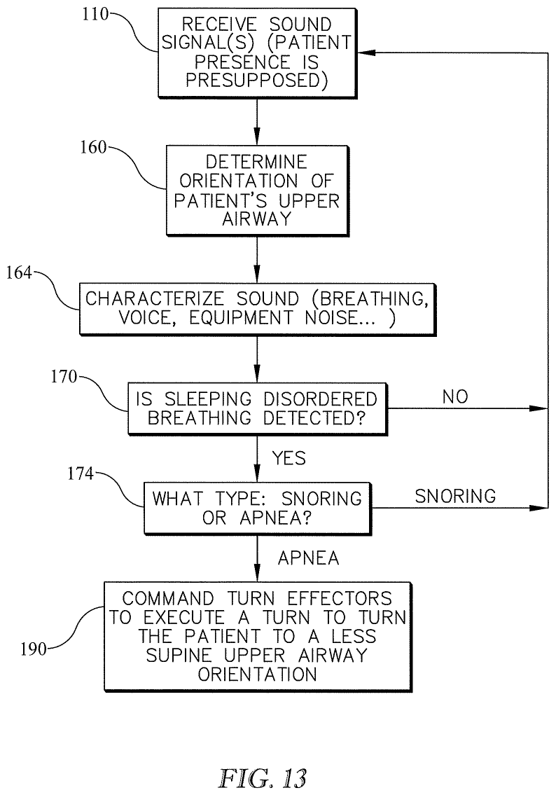

FIG. 13 is a diagram illustrating the use of acoustic information to address the actual occurrence of sleep disordered breathing, either snoring or apnea. Blocks 110 and 160 are as already described. In addition, in the interest of simplifying the diagram, the block for determining whether the patient is present or absent (block 120) is not shown. Instead, patient presence is presupposed.

At block 164 the processor characterizes the detected sounds for what they are, for example breathing, snoring, voice, equipment noise, etc. This may be accomplished by pattern matching, i.e. by comparing the detected sound profile, in the form of a time signature or sound spectrum, to profiles stored in memory 36.

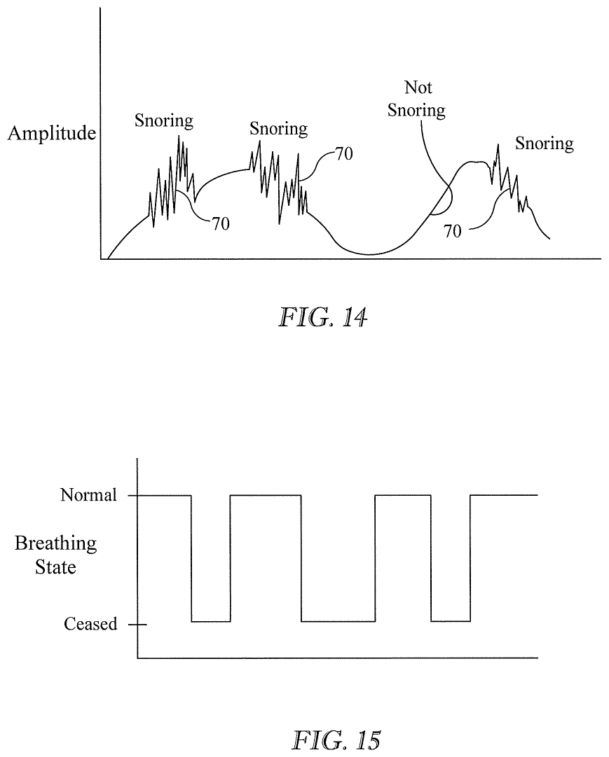

At block 170 the processor determines if the patient is experiencing sleep disordered breathing. Referring to FIG. 14, one type of sleep disordered breathing is snoring which is characterized by oscillations 70 separated by periods of normal breathing. Referring to the breathing state graph of FIG. 15, the other type of sleep disordered breathing is apnea, which is characterized by cessation of breathing. Because of the characterization of sounds at block 164, the processor can be configured to not analyze non-breathing sounds for the possibility of sleep disordered breathing at block 170, thereby reducing the burden on the processor.

If the patient is experiencing sleep disordered breathing the processor branches to block 180 and determines if the patient's breathing pattern indicates snoring or apnea. If the breathing pattern is consistent with a snoring pattern no action is taken to turn the patient. If the breathing pattern is consistent with apnea the processor advances to block 190 and commands the turn effector to turn the patient to a less supine orientation. In alternative embodiments the explicit determination of block 180 is not made. Instead block 180 is absent and a “yes” result at block 170 causes the processor to advance directly to block 190.

Continuing to refer to FIG. 13, in some embodiments the determination of the orientation of he person's upper airway (block 160) is not included. Instead, the command at block 190 is issued irrespective of any previous determination of the orientation of the person's upper airway.

FIG. 16 is a block diagram showing a variant of FIG. 13 in which turning of the patient is conditional. Blocks 110 through 170 are the same as in FIG. 13. In response to a “yes” result at block 170 the processor advances to block 180 and determines if the patient had been previously turned in order to address sleep disordered breathing. The determination can be accomplished by consulting a log stored in memory 36 which chronicles prior turning events conducted in response to a determination of sleep disordered breathing. If the patient had not been previously turned in order to address sleep disordered breathing, the processor advances to block 190 and commands the turn effector to execute a turn. If the patient had been previously turned in order to address sleep disordered breathing, the processor advances to block 195 and assesses whether the patient's airway is angularly displaced from vertical by some minimum threshold amount such as the plus or minus 15 degrees of FIG. 11. If the patient's airway orientation does not differ from vertical by more than the threshold amount the processor proceeds to block 190 and commands the turn effector to execute a turn. Otherwise the processor advances to block 200 and discontinues any further turn therapy. The processor may also cause an alert to be issued to one or more caregivers. One rationale for the discontinuance at block 200 is that because the patient's sleep disordered breathing has not responded to previous turning, he may suffer from nonpositional apnea (apnea that is not mitigated by turning the patient to a non-supine orientation).

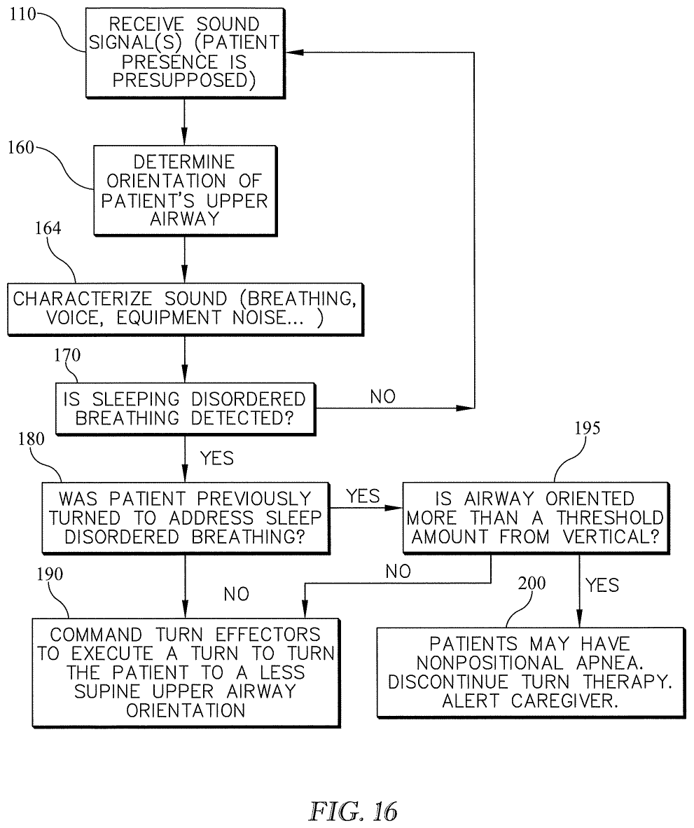

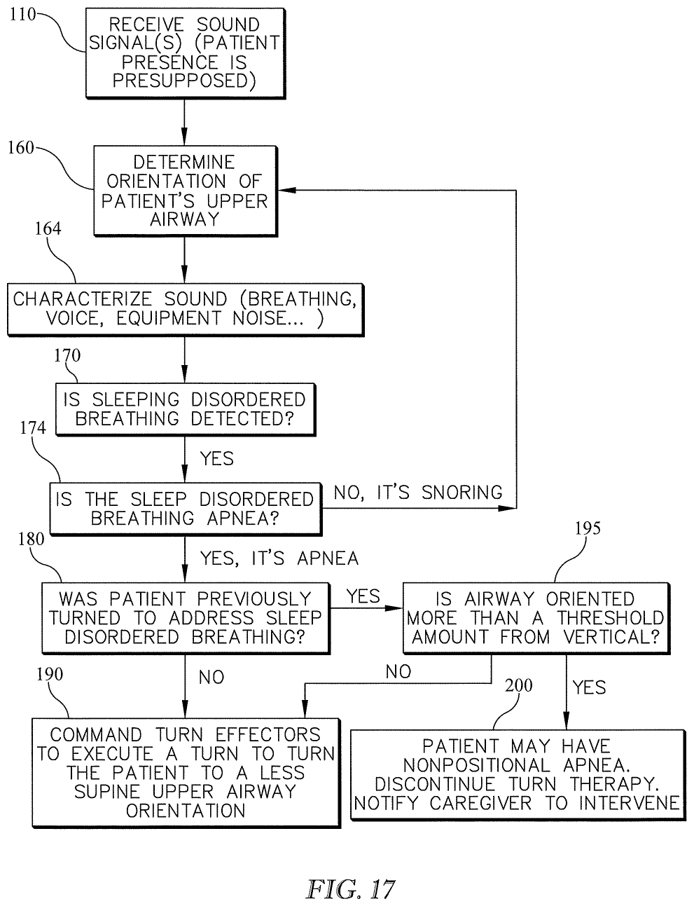

FIG. 17 is a block diagram showing a variant of FIG. 16 in which an explicit distinction is made between snoring and apnea. In response to a “yes” result at block 170 the processor advances to block 174 where it distinguishes between snoring and apnea. If snoring is detected the processor follows the “no” path back to block 160. If apnea is detected the processor follows the “yes” path to block 180, from which point operation proceeds as already explained in connection with FIG. 16.

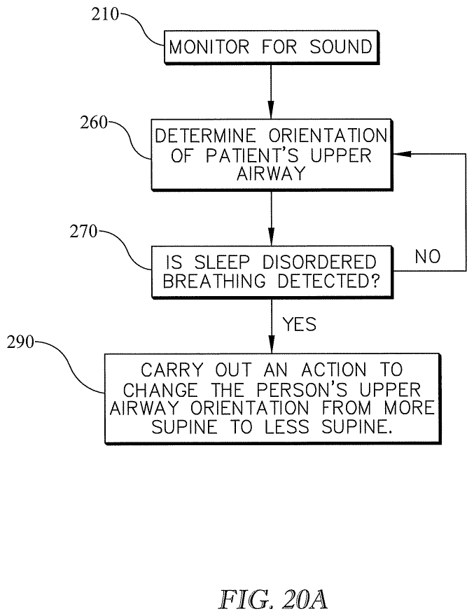

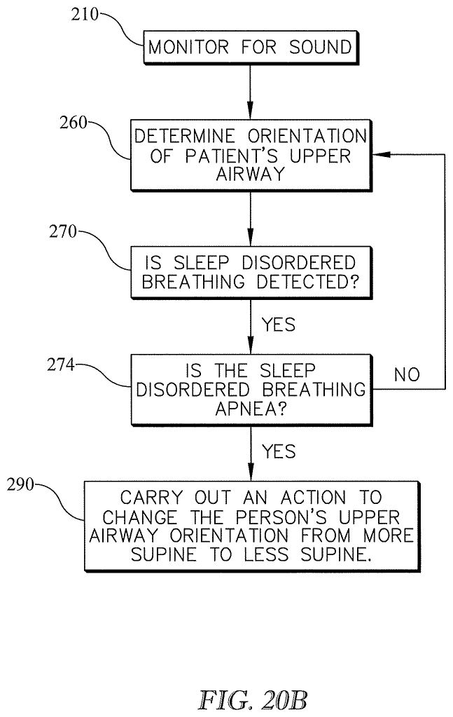

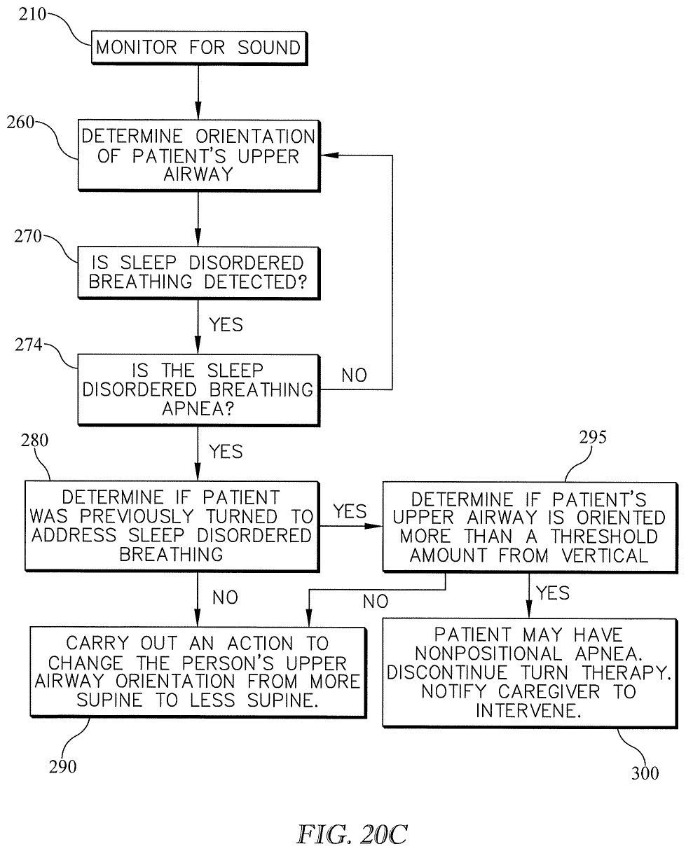

FIGS. 13, 16 and 17 all include block 164 which characterizes the detected sounds for what they are, for example breathing, snoring, voice, equipment noise, etc. However such characterization is not necessarily required because the detection of sleep disordered breathing can include the analysis of sounds other than those of breathing. The non-breathing sounds (e.g. voice or equipment noise) would not meet the criteria for being identified as sleep disordered breathing. Accordingly, block 164 can be dispensed with, and the processor can advance from block 160 directly to block 170. (The omission of a block analogous to block 164 is explicit in the method diagrams of FIGS. 20A, 20B, and 20C). As previously noted, dispensing with the action at block 164 may increase the computational burden on the processor.

FIGS. 16 and 17 both include block 160 at which the processor determines the orientation of the person's upper airway. In some embodiments that block may be relocated to the path between blocks 180 and 195.

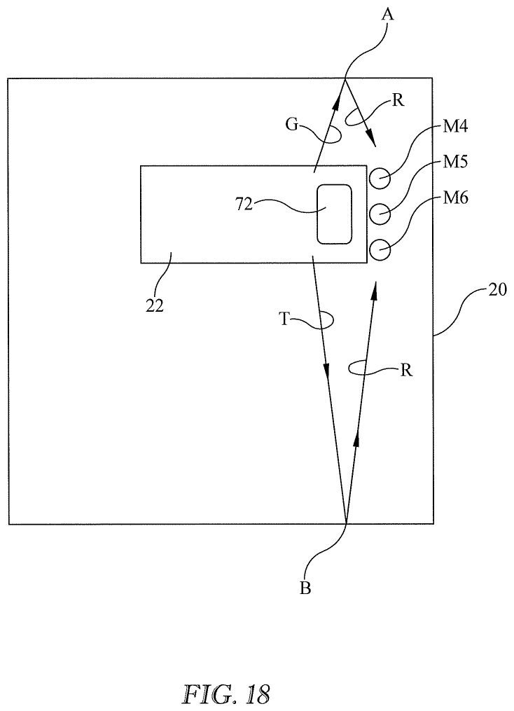

Referring now to FIG. 18, performance of the apparatus may be improved by employing a sound generator 74. The sound generator is activated to generate a test sound or signal T. Both the generated sound T and its reflections R from walls and other objects and surfaces are detected at microphones M4, M5, M6. Acting according to instructions 38, processor 36 analyzes the test and reflected signals in order to distinguish between the test signal T and its reflections R. For example the instructions may identify sounds having spectral content which are similar but are also out of phase with each other, thereby distinguishing between original sound T and reflected sound R. When a patient is being monitored the known phase relationship (or other distinguishing clues) is used to discriminate between patient generated sounds and the reflections of those sounds, thus improving detection accuracy.

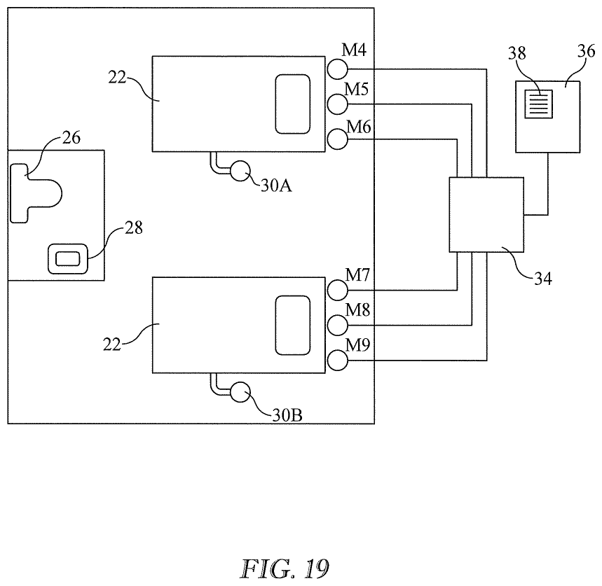

FIG. 19 is a plan view similar to FIG. 2 showing a semi-private hospital room having two beds 30A and 30B. The room includes microphones or other sound detectors, M4, M5, M6 laterally spaced from each other along the head end of bed 30A with microphone M5 laterally aligned with the longitudinally extending centerline CL of bed 30A. The room also includes microphones M7, M8, M9 laterally spaced from each other along the head end of bed 30B with microphone M8 laterally aligned with the longitudinally extending centerline CL of bed 30B. The two beds share processor 34, memory 36 and instructions 38. Processor 34, acting in accordance with instructions 38, may operate in the various ways already described.

The actions described in connection with FIGS. 5, 7, 8, 12-13, and 16-17 can be readily expressed as methods for addressing sleep disordered breathing in a person. FIG. 20A outlines an example of an associated method. At block 210 the method includes the step of monitoring for sound. At block 260 the method determines upper airway orientation of a person based on the information content of the sound. At block 270 the method determines, based on the information content of the sound, if the person is experiencing sleep disordered breathing. If the person is experiencing sleep disordered breathing the method advances to block 290 where it carries out an action to change the orientation of the upper airway of the person from an initial orientation to a target orientation. The target orientation is an orientation more conducive to relieving the sleep disordered breathing than is the initial orientation. Examples of actions to change the orientation of the person's upper airway include issuing a command to turn bladders 52, actuators 54 or other turn effectors to adjust the orientation of a bed component.

In the embodiment of FIG. 20B, step 274 distinguishes between sleep disordered breathing in the form of snoring and sleep disordered breathing in the form of apnea. Step 290 of carrying out an action is taken only if the sleep disordered breathing is apnea.

The method of FIG. 20C imposes additional preconditions on the step of carrying out an action to turn the patient to a less airway supine orientation. At block 280 the method determines if the person was previously turned to address sleep disordered breathing. If the person was not previously turned to address sleep disordered breathing the method advances to block 290 and carries out an action to turn the person so that his upper airway is in a less supine orientation. However if the person was previously turned to address sleep disordered breathing the method advances to block 295 and determines if the person's upper airway is already oriented more than a threshold amount from vertical. If not, the method proceeds to block 190. If so the method proceeds to block 300.

The foregoing description has been directed to a hospital or other formal health care setting in which each bed accommodates a single patient. However the description is applicable to other settings, such as a residential setting in which two persons share a single bed, but there is nevertheless a desire to address at least snoring and apnea.

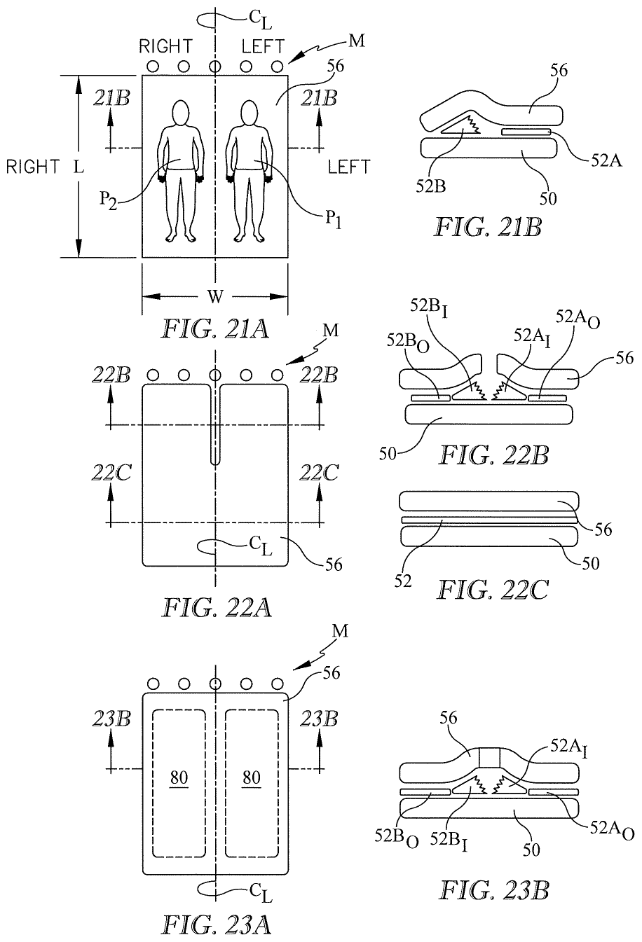

FIGS. 21A and 21B show a first embodiment of a two-person bed. The bed has a longitudinal length L and a lateral width W. An array M of five microphones extends laterally near the head end of the bed. Left and right are from the perspective of a supine occupant of the bed. The bed includes laterally continuous mattress 56 and a pair of turn effectors 52A, 52B illustrated as a bellows-like bladders. Bladder 52A resides under the left side of the mattress and accommodates a first or left side person P1. Bladder 52A is shown in its deflated state. Bladder 52B resides under the right side of the mattress and accommodates a second or right side person P2. Bladder 52B is shown in its inflated state. Each bladder has an inboard edge proximate the bed centerline CL and an outboard edge remote from the centerline. In each bladder the inboard edge is the pleated edge. As a result each bladder is unidirectional. That is, inflation of left bladder 52A turns the left person to the left (outboard) and inflation of right bladder 52B turns the right person to the right (also outboard). As a result there will be no risk of the occupants rolling toward each other in the event that both bladders are inflated.

Bladders 52A, 52B may extend substantially the entire length of the bed or may extend only part way, for example so that they reside beneath only the torso section of the mattress. Each array extends longitudinally a defined length such as the full length of the bed or part of the length of the bed so that it's longitudinal effect is limited to less than the longitudinal length of the mattress. Use of longitudinally distributed turn effector elements permit the amount of turning to be regulated as a function of longitudinal position. In another variant the turn effector is an array of two or more longitudinally distributed bladders (or other effectors such as actuators 54) provided on the left side and a similar array provided on the right side.

FIGS. 22A-22C show a second two-person embodiment. The torso section of the mattress is bifurcated. The turn effector on each side of the bed is a suite of two turn effectors—outboard and inboard turn effectors 52AO and 52AI on the left side, and outboard and inboard turn effectors 52BO and 52BI on the right side. The illustrated turn effectors are bellows-like bladders however other types of turn effectors such as mechanical actuators may be used. Both inboard bladders are shown in their inflated state. Both outboard bladders are shown in their deflated state. The pleated edge of each inboard bladder faces inboard, and the pleated edge of each outboard bladder faces outboard. As a result each occupant can be turned both left and right. As illustrated the bladders extend lenthwisely only part way along the length of the bed. In another embodiment the bladders extend substantially the full length of the bed. The members of the suite of bladders may be longitudinally distributed bladder arrays as described in connection with FIGS. 21A and 21B.

FIGS. 23A-23B show a third two-person embodiment. Each lateral side of the mattress includes a dedicated person space 80. The bladders of the turn effector bladder suites are full length bellows-like bladders, an inboard bladder and an outboard bladder on each side. Partial length bladders and/or arrays of longitudinally distributed bladders may also be used. As with the other embodiments, turn effectors other than bladders may be used.

Turn effectors in the form of longitudinally distributed elements (e.g. bladders 52; actuators 54) or two or more laterally distributed elements (which themselves may be in the form of two or more longitudinally distributed elements) as described in connection with FIGS. 21, 22 and 23 may also be used in the embodiments for a hospital or other formal health care setting.

Although this disclosure refers to specific embodiments, it will be understood by those skilled in the art that various changes in form and detail may be made without departing from the subject matter set forth in the accompanying claims.

We claim:

1-17. (canceled)

18. A system for addressing sleep disordered breathing in a person, comprising:
a) an acoustic sensor;
b) a processor;
c) a support apparatus for supporting the person, the support apparatus having a head end, a foot end, a left side and a right side, the support apparatus including or receptive to receiving a turn effector; and
d) processor executable instructions which, when executed by the processor, cause the system to:
1) determine, based on the information from the acoustic sensor, if the person is experiencing sleep disordered breathing; and
2) if the person is experiencing sleep disordered breathing, command operation of the turn effector to act on a component of the support apparatus thereby changing the orientation of the upper airway of the person from an initial orientation to a target orientation wherein the target orientation is an orientation more conducive to relieving the sleep disordered breathing than is the initial orientation.

19. The system of claim 18 wherein the processor also causes determination of the upper airway orientation of the person based on information from the acoustic sensor.

20. The system of claim 18 wherein the executable instructions, when executed by the processor, cause the system to distinguish between sleep disordered breathing in the form of snoring and sleep disordered breathing in the form of apnea and to command operation of the turn effector only if the sleep disordered breathing is apnea.

21. The system of claim 18 wherein the processor executable instructions, when executed by the processor, cause the system to carry out the action of commanding operation of the turn effector only if:
A) the person had not been previously turned in response to sleep disordered breathing, or
B) the person had been previously turned in response to sleep disordered breathing and the person's airway is not oriented more than a threshold amount away from vertical.

22. The system of claim 21 wherein the instructions, when executed by the processor, cause an alert to be issued if the conditions A and B are both unsatisfied.

23. The system of claim 21 wherein the processor executable instructions, when executed by the processor, cause the system to determine if the sleep disordered breathing is apnea and, if so, to refrain from commanding operation of the turn effector.

24-35. (canceled)

36. The system of claim 18 wherein the system determines if the person is experiencing sleep disordered breathing by determining upper airway orientation of the person based on information from the acoustic sensor.

37. The system of claim 36 wherein the initial orientation is a more supine orientation and the target orientation is a less supine orientation.

38. The system of claim 36 wherein turn effector acts on the component by re-orienting the component from a first orientation to a second orientation, and wherein the difference between the first and second orientations is a function of the first orientation.

39. The system of claim 36 wherein the actions of determining upper airway orientation and commanding operation of the turn effector are continuously ongoing.

40. The system of claim 36 wherein the processor executable instructions, when executed by the processor, cause the system to determine sleep state of the person before determining the upper airway orientation of the person.

41. The system of claim 40 wherein the processor executable instructions, when executed by the processor, cause the system to determine person presence or lack thereof before determining the sleep state of the person.
